(19) United States
(12) Patent Application Publication
(10) Pub. No.: US 2014/0142168 A1
(43) Pub. Date: May 22, 2014

(54) METHODS AND PHARMACEUTICAL COMPOSITIONS FOR THE TREATMENT OF HORMONE-REFRACTORY PROSTATE CANCERS

(71) Applicants: Chantal Barthomeuf, Paris (FR); Philippe Chollet, Clermont-Ferrand (FR); Eloise Planchat, Clermont-Ferrand (FR); Hervé Cure, Reims (FR)

(72) Inventors: Chantal Barthomeuf, Paris (FR); Philippe Chollet, Clermont-Ferrand (FR); Eloise Planchat, Clermont-Ferrand (FR); Hervé Cure, Reims (FR)

(73) Assignees: CENTRE JEAN PERRIN, Clermont-Ferrand (FR); Institut National de la Sante et de la Recherche Medicale (INSERM), Paris (FR); UNIVERSITE D'AUVERGNE CLERMONT, Clermont-Ferrand (FR)

(21) Appl. No.: 14/114,223

(22) PCT Filed: Apr. 27, 2012
(86) PCT No.: PCT/EP12/57740

§ 371 Date: Feb. 4, 2014

(30) Foreign Application Priority Data
Apr. 28, 2011 (EP) 11305499.3

(51) Int. Cl. A61K 31/337; A61K 31/12

(52) U.S. Cl. CPC A61K 31/337; A61K 31/12
USPC 514/449

(57) ABSTRACT

The present invention relates to a combination of a curcuminoid and a taxane for use in the treatment of a hormone-refractory prostate cancer (HRPC) in a patient in need thereof.

### FIELD OF THE INVENTION

The present invention relates to the field of hormone-refractory prostate cancer treatment.

### BACKGROUND OF THE INVENTION

In 2007, prostate cancer has been identified as the first cause of cancer and the 5th cause of death in man from developed countries. The frequency of patients presenting at each stage of disease has changed remarkably with introduction of prostate specific antigen (PSA) screening in the early 1990s.

Approximately 30-35% of patients with prostate cancer will present with regional or metastatic tumors, while an additional 25% will develop metastases in the course of the disease. Patients with metastatic disease are initially treated with hormone therapy such as luteinizing hormone releasing hormone (LHRH) agonists or antagonists, diethylstilbestrol (DES), orchiectomy, and/or anti-androgens. The development of hormonal resistance occurs in most patients after androgen deprivation. The term “hormone-refractory prostate cancer” (HRPC) is used by physicians to describe prostate cancer disease that progresses despite castrate levels of serum testosterone.

The median time to progression to HRPC is 18 months from the time of initiation of hormonal therapy against prostate cancer. Responses to current second line hormonal therapies are temporary and do not impact upon survival. The median survival after developing HRPC has been 12 to 18 months, and until recently, there was no clearly effective systemic treatment for this condition.

With recent advances in the understanding of HRPC, novel treatment regimens were identified. HRPC is much less sensitive to cytostatics than its “sister” tumor breast cancer for example anthracyclines, alkylating drug or vinca alcaloïds are not effective in most cases. However taxanes appeared to possess clinical activity leading to market authorization.

For example, uses of taxanes such as paclitaxel, docetaxel, larotaxel or cabazitaxel have been investigated. For example, docetaxel is the reference first-line of treatment of HPRC. Docetaxel is administered with prednisolone with or without other therapeutics. Docetaxel induces a PSA response in 45% of treated patients. However, the objective tumor response is limited to 12%. Accordingly, there is a need for improving clinical efficacy of docetaxel, and more generally, taxanes in the treatment of HPRC.

### SUMMARY OF THE INVENTION

The present invention relates to a combination of a curcuminoid and a taxane for use in the treatment of a hormone-refractory prostate cancer (HRPC) in a patient in need thereof.

### DETAILED DESCRIPTION OF THE INVENTION

The inventors have demonstrated that cucurminoids enhance significantly the response of a reference taxane (e.g; docetaxel) in the treatment of HRPC. More particularly, the inventors have conducted a phase II study to assess the response of HPRC to docetaxel/curcuminoid combination. A PSA response was observed in 17 of the 29 evaluable patients (59%). The responses were complete (4 patients) or partial (13 patients) and were observed rapidly (before the 3rd cycle) in 15 patients. Fifteen of 30 patients had measurable or evaluable lesions (RECIST criteria), with 6 (40%) partial responses and 9 (60%) stable disease. The median PSA TTP was 6.00 months. The response is further observed before the appearance of a resistance to docetaxel. Finally, haematological toxicity is lower that the one one observed without curcuminoids: the most common grade-3-4 hematological toxicity was neutropenia and 5 patients presented with grade-2 anemia. Accordingly, combination of curcuminoids with taxanes present an improved therapeutic efficacy in the management of HPRC. To explain the observations, the inventors have further investigated the mechanisms of action and demonstrate that the clinical endpoints are not explained by the well-known effects of curcuminoid on multidrug resistance (i.e Pgp resistance) as described in the International Patent Application Publication WO2010/115852. Surprisingly, the inventors indeed show that curcuminoids may enhance the level of objective response and delay of resistance acquisition through the down regulation of telomerase activity.

Accordingly, the present invention relates to a combination of a curcuminoid and a taxane for use in the treatment of a hormone-refractory prostate cancer (HRPC) in a patient in need thereof.

As used herein, the term “taxane ” has its general meaning in the art and refers to a well known class of chemotherapeutic agents that include but are not limited to docetaxel paclitaxel, larotaxel or cabazitaxel. The taxanes are diterpenes that were originally derived from plants of the genus Taxus (yews).

As used herein the term “docetaxel” or “DTX” refers to (2R,3S)-N-carboxy-3-phenylisoserine, N-tert-butyl ester, 13-ester with 5, 20-epoxy-1,2, 4, 7, 10, 13-hexahydroxytax-11-en-9-one 4-acetate 2-benzoate, trihydrate. (CAS number: 114977-28-5).

As used herein the term “paclitaxel” refers to (2α,4α,5β,7β,10β,13α)-4,10-bis(acetyloxy)-13-{[(2R,3S)- 3-(benzoylamino)-2-hydroxy-3-phenylpropanoyl]oxy}- 1,7-dihydroxy-9-oxo-5,20-epoxytax-11-en-2-yl benzoate.

As used herein, the term “larotaxel” refers to (2α,3ξ,4α,5β,7α,10β,13α)-4,10-bis(acetyloxy)-13-({(2R,3S)-3-[(tert-butoxycarbonyl)amino]-2-hydroxy-3-phenylpropanoyl}oxy)-1-hydroxy-9-oxo-5,20-epoxy-7,19-cyclotax-11-en-2-yl benzoate

As used herein, the term “cabazitaxel” refers to 1S,2S,3R,4S,7R,9S,10S,12R,15S)-4-(Acetyloxy)-15-{[(2R,3S)-3-{[(tert-butoxy)carbonyl]amino}-2-hydroxy-3-phenylpropanoyl]oxy}-1-hydroxy-9,12-dimethoxy-10,14,17,17-tetramethyl-11-oxo-6-oxatetracyclo[11.3.1.03,10.04,7]heptadec-13-ene-2-yl benzoate.

In a particular embodiment, the taxane may represent the first line of treatment in HRPC (e.g. docetaxel) or the second line of treatment in HPRC (e.g. cabazitaxel).

Typically, the taxane is administered in a therapeutically effective amount to the patient. Generally speaking, a “therapeutically effective amount”, or “effective amount”, or “therapeutically effective”, as used herein, refers to that amount which provides a therapeutic effect for a given condition and administration regimen. This is a predetermined quantity of active material calculated to produce a desired therapeutic effect in association with the required additive and diluent; i.e., a carrier, or administration vehicle. Further, it is intended to mean an amount sufficient to reduce and most preferably prevent a clinically significant deficit in the activity, function and response of the host. Alternatively, a therapeutically effective amount is sufficient to cause an improvement in a clinically significant condition in a host. As is appreciated by those skilled in the art, the amount of a compound may vary depending on its specific activity. Suitable dosage amounts may contain a predetermined quantity of active composition calculated to produce the desired therapeutic effect in association with the required diluents; i.e., carrier, or additive.

Typically the taxane may be administered to the patient at a concentration accepted by the regulatory agencies. Typically docetaxel could be administered intravenously at the concentration accepted by EMEA for treatment of patients with HPRC: dose of [75 mg/m²] every 21 days, for 6 cycles. However, lower doses of taxane than those recommend by the regulatory agencies may also be used. For example about half concentration of the recommended doses may be administered to the patient (e.g. 40 mg/m² for docetaxel weekly).

The taxane of the invention is typically administered to patient through the mode of administration authorized by the regulatory agencies. For example, the taxane is administered intravenously at concentration accepted by EMEA for treatment of patients with HPRC.

The term “curcuminoid” encompasses natural curcumin (diferuloylmethane, feruloyl (1E,6E)-1,7-bis (4-hydroxy-3-methoxyphenyl)-1,6-heptadiene-3 ,5-dione) and structurally-derived compounds. Curcuminoids contains three major derivative coumpounds: curcumin (diferuloylmethane, curcumin I, CAS number: 458-37-7), demethoxycurcumin (4-hydroxycinnamoyl(feroyl)methane, curcumin II, CAS number: 24939-17-1), and bisdemethoxycurcumin (Bis(4-hydroxycinnamoyl)methane, curcumin III, CAS number: 24939-16-0). Those compounds have the formula as follows:

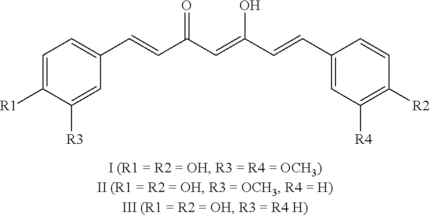

Curcuminoids can exist in at least two tautomeric forms, keto and enol. They may differ from curcumin by the number of ethylene units (one or two), the number of conjugated aromatic rings (one or two), the number of phenol moeties on each ring (one or two), the number and nature of additional substituents (usually alkyloxy derivatives, methoxy in curcumin) or by a combination of the foregoing.

In a particular embodiment, the curcuminoid may be administered to the patient in a curcuminoid extract containing curcumin as main component and demethoxycurcumin and bisdemethoxycurcumin as minor components.

In a particular embodiment, curcuminoids of the invention is administered to the patient through any route but preferably orally. Suitable unit oral-route forms include but are not limited to tablets, capsules, powders, granules, oral suspensions or solutions, sublingual and buccal administration forms. Typically, the curcuminoid compound of the invention is administered through oral capsules, one, two or three-times a day. The daily dose of the curcuminoid composition is typically about 5 g administered as individual buccal administration forms containing about 425 mg of curcuminoids (containing at least 300 mg of curcumin) 3-fold/day at breakfast, lunch and diner-time.

In a particular embodiment, curcuminoids of the invention is administered before, concomitantly and after the administration of the taxane. In a preferred embodiment, the curcuminoid is administered daily for a period of several days during which a single dose of the taxane is administered. According to this embodiment, the curcuminoid compound is administered for 7 days. In a particular embodiment, this cycle of administration (i.e. a period of several days during which the curcuminoid compound is administered daily and a single dose of the taxane is administered) may be repeated. The cycles of administration may be separated by a period during no therapeutic agent is administered. In a particular embodiment the cycle may be repeated 6 times every 21 days.

In a particular embodiment, the combination may further comprise a corticosteroid such as prednisolone.

In a particular embodiment, the active ingredients of the invention are used in combination with one or more pharmaceutically acceptable excipient or carrier. By “physiologically acceptable excipient or carrier” is meant solid or liquid filler, diluents or substances that may be safely used in oral or systemic administration. Depending on the particular route of administration, a variety of pharmaceutically acceptable carriers well known in the art include lipid oil or lipid oil derivatives (cremophor), combination with other lipids, solid or liquid fillers, diluents, hydrotropes, surface active agents, and/or encapsulating substances. Pharmaceutically acceptable carriers for systemic administration that may be incorporated in the composition of the invention include sugar, starches, cellulose, vegetable oils or natural lipids including cremophor or soya phospholipids, buffers, polyols, alginic acid. Representative carriers include membrane lipids, acacia, agar, alginates, hydroxyalkylcellulose, hydroxypropyl methylcellulose, carboxymethylcellulose, carboxymethylcellulose sodium, carrageenan, powdered cellulose, guar gum, cholesterol, gelatin, gum agar, gum arabic, gum karaya, gum ghatti, locust bean gum, octoxynol 9, oleyl alcohol, pectin, poly(acrylic acid) and its homologs, polyethylene glycol, polyvinyl alcohol, polyacrylamide, sodium lauryl sulfate, poly(ethylene oxide), polyvinylpyrrolidone, glycol monostearate, propylene glycol monostearate, xanthan gum, tragacanth, sorbitan esters, stearyl alcohol, starch and its modifications. Suitable ranges vary from about 0.5% to about 1%. For formulating the active ingredients according to the invention, the one skilled in the art will advantageously refer to the last edition of the European pharmacopoeia or the United States pharmacopoeia. Preferably, the one skilled in the art will refer to the fifth edition “2005” of the European Pharmacopoeia, or also to the edition USP 28-NF23 of the United States Pharmacopoeia.

In a particular embodiment, said the combination of the patient is administered to HRPC patient who are considered as sensitive to the taxane that is administered to the patient. As used herein, by “sensitive” is meant that the prostate cancer cells of said patient respond (e.g., by a retardation in cell growth and/or cell division, an abrogation in cell growth and/or cell division, or cell death) to the taxane that is administered to the patient. Resistance could be easily detected by the skilled man in the art. For example, the detection of Pgp over expression in tumor cells is associated with resistance.

The invention will be further illustrated by the following figures and examples. However, these examples and figures should not be interpreted in any way as limiting the scope of the present invention.

### EXAMPLE 1

### Pilot Phase II Study with Docetaxel in Combination with Curcuminoids in Patients with Hormone Resistant Prostate Cancer (HRPC).

Methods:

Docetaxel was given (75mg/m2+prednisolone, every 3 weeks for 6 cycles) in combination with curcuminoids orally (purity of 85%) at the dose of 6g/day according to previous phase I schedule of administration (7 days/cycle). The primary endpoint was the response rate assessed by clinical, biological and paraclinical examinations. The secondary endpoints included safety, time to progression (TTP) and compliance.

Inclusion criteria were
Age>18
WHO performance status 0-2
Life expectancy≧3 months
Patients receiving androgen-suppressive therapy in the form of chirurgical castration by orchiectomy or pulpectomy, or medical by LHRH agonist or antagonist with or without anti-androgen or all treatment blocking non gonadic testosterone fraction
Resulting to testosteronemia<0,5 ng/ml
Histologically confirmed adenocarcinomia of prostate cancer and documented hormone independent metastatic disease—defined by: objective progression with at least one measurable lesion and/or evaluable lesion according to RECIST criteria and/or a rise in PSA level (“rising PSA”)
Total bilirubin≦upper limit of normal (ULN).
AST and ALT≦1.5 times ULN. Alkaline phosphatase≦2.5 times ULN.
Serum creatinine<140 !mol/L or creatinine clearance>60 mL/ min.
Neutrophil count>2.109 L-1.
Platelet count≧100,000/mm3.
Hemoglobin≧10 g/dL
Not previous chemotherapy, except Estracyt
No liver, kidney or heart failure link to treatment
No malabsorption syndrome or disease significantly affecting gastrointestinal function
Prior radiotherapies are permitted within four weeks of the first study treatment and must be<25% of the bone marrow, and all adverse events must be resolved
Prior surgery are permitted.

Exclusion Criteria:
Age<18
History of psychiatric disorders including psychotic disorder, dementia or seizures that would prohibit the understanding, observance and giving of informed consent
Previous or concomitant other malignancies except basal or squamous cell carcinoma of the skin or other cancer curatively treated with surgery and/or radiotherapy
Patients should not have symptomatic brain metastasis
Concurrent severe and/or uncontrolled co-morbid medical condition
Malabsorption syndrome or disease significantly affecting gastro-intestinal function or major resection of the stomach, proximal small bowel or grade>2 dysphagia
Patients with uncontrolled infection
History of significant neurologic (i.e. peripheral neuropathy grade>2 using NCI-CTC criteria v3.0)
Patients should not have received NSAIDs or COX2 inhibitors within the three weeks prior to starting the study
Treatment with any investigational drug within 30 days prior to registration
Patients should not have current regimen containing dietary phytonutrients*

Results:

Thirty patients included (October 2009 to November 2010), had a mean age of 69 years (range 58-83). Of 169 cycles, 150 (89%) were completed with perfect compliance. Twenty six patients received the 6 courses and 4 withdrew prematurely. No patient withdrew for toxicity. The most common grade-3-4 hematological toxicity was neutropenia and 5 patients presented with grade-2 anemia. For non-hematological toxicity, 4 grade-3 and 23 grade-2 adverse events were observed: mainly asthenia, diarrhea and ungueal toxicity. A PSA response was observed in 17 of the 29 evaluable patients (59%). The responses were complete (4 patients) or partial (13 patients) and were observed rapidly (before the 3rd cycle) in 15 patients. Fifteen of 30 patients had measurable or evaluable lesions (RECIST criteria), with 6 (40%) partial responses and 9 (60%) stable disease. The median PSA TTP was 6.00 months, the median TTP on targets was 6.87 months (n=9/15).

The increase in PSA is thus limited (59% vs of 45% observed in the reference randomized study) but the increase in objective responses is high (40% instead of 12% observed in the reference randomized study).

Conclusions:

In HRPC patients, combination with curcuminoids tends to improve the response rate of docetaxel in terms of both PSA level and objective responses. This justify further randomized trials.

### EXAMPLE 2

### Biological Data

It was deduced from the investigator data that 1 h after administration of 75 mg/m2 of DTX, blood levels of DTX can reach 3.75 μM. Based on previous data, blood levels reached 1.77 μM after oral ingestion of 8,000 g of curcumin. We thereby expected that 1 h after ingestion of 2,000 g of curcumin, the blood levels of curcumin in patients can vary between 0.1 and 0.45 μM.

The PC3 line is a human p-53 defective HRPC line displaying high SphK1 activity. To mimic the effect of treatment, 10,000 PC3 cells were seeded in each well of 24-well plates and maintained in DMEM containing 10% FBS (complete medium) overnight. Then they were:
1) treated for 2 h by 3.75 μM of DTX in DMSO and maintained in drug-free medium (DFM) for 70 h (Total duration of treatment: 72 h).
2) repeatedly treated by 0.25 μM of curcumin for 3 days. Indeed, in these experiences PC3 cells were treated the first day for 2 h at 7 h AM and maintained in DFM until 12 h AM (5h). At this time, the medium was replaced by new complete medium containing 0.25 μM of curcumin. Replacement of medium by new complete medium containing 0.25 μM curcumin was done at 19 h the first day and at 7 h, 12 h and 19 h the second and third days
3 ) repeatedly treated by curcumin (0.25 μM) for 2 days before a 2 h treatment by DTX. The third day, DTX was added in new complete medium, 2 h after the last treatment by curcumin. As previously, the cells were maintained in DFM for 70 h in DFM after DTX treatment.

PC3 survival was determined at the end of treatment 1 and 3. Telomerase activity was determined in survived cells by Trap assay at the end of treatment 1, 2 and 3.

At the end of DTX treatment, the number of survived cells was increased from 90±5 (DTX singly) to 93±4% (DTX+curcumin pretreatment). Thus curcuminoid did not improved significantly the toxicity of this very high dose of DTX supporting a similar level of response of patients treated by DTX singly after the 1 cure of DTX

We hypothesized that curcuminoids may enhance the level of objective responses and delay of acquisition of resistance through interaction with telomerase activity. The telomerase activity was significantly increased in cells surviving DTX treatment evidenced that DTX selected rapidly resistant sub-populations. Repeated exposure to 0.25 uM of curcumin attenuate the increase induced by lower doses of DTX but did not attenuate significantly the increase in telomerase activity induced by this high dose of DTX but they significantly lowered the telomerase activity of untreated cells. Data suggest that altogether pre- and post-exposure to curcuminoids may induce (i) a partial [symbol] redifferentiation [symbol] of HRPC cells lowering the rate of proliferation (and accordingly the invasiveness) of HRPC tumors and (ii) may delay the formation of resistant sub-populations and, thus the acquisition of resistance to DTX.

### REFERENCES

Throughout this application, various references describe the state of the art to which this invention pertains. The disclosures of these references are hereby incorporated by reference into the present disclosure.

1. A method for the treatment of a hormone-refractory prostate cancer (HRPC) in a patient in need thereof comprising administering to the patient a combination of a curcuminoid and a taxane.

2. The method according to claim 1 wherein the taxane is selected from the group consisting of docetaxel, paclitaxel, larotaxel or cabazitaxel.

3. The method according to claim 2 wherein said taxane is docetaxel.

4. The method according to claim 1 wherein the curcuminoid is selected from the group consisting of curcumin demethoxycurcumin and bisdemethoxycurcumin.
